(19) United States
(12) Patent Application Publication
(10) Pub. No.: US 2025/0041215 A1
(43) Pub. Date: Feb. 6, 2025

(54) MULTIDOSE PRESERVATIVE-FREE (MDPF) OPHTHALMIC DOSE DELIVERY WITH ADDITION OF FORMULATION SURFACTANTS

(71) Applicant: ALCON INC., Fribourg (CH)

(72) Inventors: Hongzhu Liao, Fort Worth, TX (US); Randall Renato Kolega, Arlington, TX (US)

(21) Appl. No.: 18/786,773

(22) Filed: Jul. 29, 2024

Related U.S. Application Data
(60) Provisional application No. 63/517,360, filed on Aug. 3, 2023.

(51) Int. Cl. A61K 9/08; A61K 9/00; A61K 31/335; A61K 31/4168; A61K 47/34; A61K 47/38

(52) U.S. Cl. CPC A61K 9/08; A61K 9/0048; A61K 31/335; A61K 31/4168; A61K 47/34; A61K 47/38

(57) ABSTRACT

The present disclosure generally relates to ophthalmic compositions and multidose systems for topically applying compositions to a surface, e.g. an eye surface. Multidose systems of the present disclosure can include a multidose preservative-free (MDPF) eye dropper bottle system and an ophthalmic composition having an therapeutic agent, a viscosifying agent having a concentration of about 0.01% w/v to about 5% w/v, and a surfactant having a concentration of about 0.005% w/v to about 7% w/, wherein the composition lacks a preservative.

### BACKGROUND

### Field

Aspects of the present disclosure generally relate to ophthalmic compositions.

### Description of the Related Art

Aqueous, multi-dose pharmaceutical compositions have been formulated so as to have sufficient antimicrobial activity to satisfy the preservation efficacy requirements of the United States Pharmacopeia (“USP) and analogous guidelines in other countries, without requiring a conventional antimicrobial preservative, e.g., benzalkonium chloride, polyquaternium-1, sodium perborate, or chlorine-containing agents.

Many pharmaceutical compositions are sterile for use. Examples of such compositions include various types of compositions that are applied either directly to the eye, e.g., artificial tears, irrigating solutions, and drug products, or are applied to devices that will come into contact with the eye, e.g., contact lenses.

The foregoing types of compositions can be manufactured under sterile conditions via procedures that are well known to those skilled in the art. However, once the packaging for a product is opened, such that the composition contained therein is exposed to the atmosphere and other sources of potential microbial contamination, the sterility of the composition may be compromised. Due to the frequent, repeated exposure of multidose products to the risk of microbial contamination, a multidose preservative-free (MDPF) eye dropper bottle system that prevents or reduces the risk of microbes reaching a pharmaceutical composition within a container may be employed. Examples of commercially available MDPF systems include the Novelia® preservative-free multidose eye dropper bottle from Nemera and the Ophthalmic Squeeze Dispenser preservative-free multidose eye dropper bottle available from Aptar Pharma. The Novelia, Aptar, and other MDPF systems are reviewed in Campolo, et al., “A Review of the Containers Available for Multi-Dose Preservative-Free Eye Drops,” Biomed J Sci & Tech Res 45(1)-2022.

Unfortunately, due to the functionality of MDPF systems, excess pharmaceutical composition is often dosed during each delivery, creating a residue that forms at the nozzle of the MDPF system when using certain viscosifying agents that enhance bioavailability. The residue causes increased and inconsistent dosing, known as spreading onto the top of the MDPF nozzle which has been identified across a wide range of MDPF systems. Moreover, the residue may serve as a source of contamination at the nozzle tip.

Therefore, there is a need in the art for improved multidose pharmaceutical compositions without requiring antimicrobial preservatives and that can be administered by a wider array of MDPF eye dropper bottle systems while maintaining sufficient viscosity to reduce or prevent droplet spreading.

### SUMMARY

The present disclosure generally relates to ophthalmic compositions and multidose eye dropper bottle systems for applying compositions to a surface, e.g. an eye surface. Multidose systems of the present disclosure can include a multidose preservative-free (MDPF) eye dropper bottle and an ophthalmic composition having a therapeutic agent, a viscosifying agent having a concentration of 0.01% w/v to 5% w/v, and a surfactant having a concentration of 0.005% w/v to 7% w/v, in which the ophthalmic composition lacks a preservative.

Aspects of the present disclosure also relate to an ophthalmic composition having an therapeutic agent having a concentration of 0.01% w/v to 1% w/v, a viscosifying agent having a concentration of 0.01% w/v to 0.8% w/v, and a surfactant having a concentration of 0.01% w/v to 1% w/v, in which the multidose preservative-free system is substantially free of benzalkonium chloride or other conventional antimicrobial preservative.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope.

FIG. 1 shows an end tip of a MDPF system, according to embodiments of the disclosure.

FIGS. 2A to 2D show alternative embodiments of an air-permeable membrane, according to embodiments of the disclosure. FIG. 2A shows a first air-permeable membrane. FIG. 2B shows a second air-permeable membrane. FIG. 2C shows a third air-permeable membrane. FIG. 2D shows a fourth air-permeable membrane.

FIG. 3 shows a graph of drop size over a number of drops dispensed of ophthalmic compositions A3 and A5, according to embodiments of the disclosure.

FIG. 4 shows a graph of surface tension of the ophthalmic composition A or B over a concentration of surfactant added to ophthalmic composition A or B, according to embodiments of the disclosure.

FIG. 5 shows a graph of surface tension of the ophthalmic composition A or B over a concentration of surfactant added to ophthalmic composition A or B, according to embodiments of the disclosure.

FIG. 6 shows a graph of a drop size dispensed of the ophthalmic composition A or B over a surface tension of ophthalmic composition A or B, according to embodiments of the disclosure.

FIG. 7 shows a graph of drop size over a number of drops dispensed of ophthalmic composition B, according to embodiments of the disclosure.

FIG. 8 shows a graph of drop size over a number of drops dispensed of ophthalmic composition B containing the surfactant tyloxapol, according to embodiments of the disclosure.

FIG. 9 shows a graph of drop size over a number of drops dispensed of ophthalmic composition B containing the surfactant polysorbate 80, according to embodiments of the disclosure.

FIG. 10 shows a graph of drop size over a number of drops dispensed of ophthalmic composition B containing the surfactant polyoxyl 40 stearate, according to embodiments of the disclosure.

FIG. 11 shows a graph of drop size over a number of drops dispensed of ophthalmic composition A with a preservative, according to embodiments of the disclosure.

FIGS. 12A and 12B shows a graph of drop size over a number of drops dispensed of ophthalmic composition A with or without flicking, according to embodiments of the disclosure.

FIGS. 13A-13C show graphs of average drop size dispensed of ophthalmic composition A relative to concentration of a surfactant, according to embodiments of the disclosure. FIG. 13A shows a graph of drop size dispensed of ophthalmic composition A relative to concentration of tyloxapol. FIG. 13B shows a graph of drop size dispensed of ophthalmic composition A relative to concentration of polysorbate 80. FIG. 13C shows a graph of average drop size dispensed of ophthalmic composition A relative to concentration of polyoxyl 40 stearate.

### DETAILED DESCRIPTION

To facilitate understanding, identical reference numerals have been used, where possible, to designate identical elements that are common to the figures. It is contemplated that elements and features of one embodiment may be beneficially incorporated in other embodiments without further recitation.

As used herein, “the ophthalmic composition lacks a preservative” means the composition is substantially free (i.e., has no more than about 0.001% w/v) of conventional antimicrobial preservatives. “Conventional antimicrobial preservatives” means preservative ingredients having a primary function of providing preservative efficacy in topically administrable ophthalmic compositions, such as benzalkonium chloride, polyquaternium-1, sodium perborate, chlorobutanol, methyl paraben, and thimerosal. Preservative enhancing agents are not considered to be preservative ingredients having a primary function of providing preservative efficacy, and examples of preservative enhancing agents include sodium borate, boric acid, citrates, tartaric acid, disodium EDTA, BHA, BHT, sodium metabisulfites, tocopherol, ascorbic acid, and sorbic acid.

Aspects of the present disclosure generally relate to ophthalmic compositions and multidose eye dropper bottle systems for topically applying compositions to a surface, e.g. an eye surface. Multidose systems of the present disclosure can include a multidose preservative-free (MDPF) eye dropper bottle or container system and an ophthalmic composition having an therapeutic agent, a viscosifying agent having a concentration of 0.01% w/v to 5% w/v, and a surfactant having a concentration of 0.005% w/v to 7% w/v, wherein the ophthalmic composition lacks a preservative. It has been discovered that a surfactant present in the ophthalmic composition maintains the bioavailability benefits of the viscosifying agent while concurrently reducing spreading on the eye dropper bottle tip. The surfactant causes the ophthalmic composition to be delivered at a consistent dosage/droplet size when using an MDPF container, reducing the amount of droplet spreading. Additionally, due to the enhanced control of the dosing when using the surfactant, less overall ophthalmic composition can be used in order to provide sufficient dosing amounts of therapeutic agent, resulting in a lower cost to the consumer for the ophthalmic composition.

Aspects of the present disclosure also relate to an ophthalmic composition having an therapeutic agent having a concentration of 0.01% w/v to 1% w/v, a viscosifying agent having a concentration of 0.01% w/v to 0.8% w/v, and a surfactant having a concentration of 0.01% w/v to 1% w/v, and more preferably 0.01 to 0.5% w/v, wherein the ophthalmic composition lacks a preservative.

Aspects of the present disclosure also relate to methods of forming a multidose system for topical ophthalmic administration including dispensing an ophthalmic composition having an therapeutic agent, a viscosifying agent having a concentration of 0.01% w/v to 5% w/v, and a surfactant having a concentration of 0.005% w/v to 7% w/v, but lacking a preservative, in a MDPF dropper bottle system to provide enhanced control of dosing.

### Ophthalmic Composition

The ophthalmic pharmaceutical compositions of the present disclosure may contain various types of therapeutic agents. Examples of possible therapeutic agents include beta blockers (e.g., timolol, betaxolol, levobetaxolol, carteolol, levobunolol, and propranolol), carbonic anhydrase inhibitors (e.g., brinzolamide and dorzolamide), alpha-1 antagonists (e.g., nipradolol), alpha-2 agonists (e.g. apraclonidine, and brimonidine), miotics (e.g., pilocarpine and epinephrine), prostaglandin analogs (e.g., latanoprost, travoprost and unoprostone), hypotensive lipids (e.g., bimatoprost), neuroprotectants (e.g., memantine), serotonergics e.g., 5-HT agonists, such as S-(+)-1-(2-aminopropyl)-indazole-6-ol), anti-angiogenesis agents (e.g., anecortave acetate), anti-infective agents (e.g., quinolones, such as moxifloxacin and gatifloxacin, and aminoglycosides, such as tobramycin and gentamicin), non-steroidal and steroidal anti-inflammatory agents (e.g., prednisolone, dexamethasone, lotoprednol, suprofen, diclofenac and ketorolac), growth factors (e.g., EGF), immunosuppressant agents (e.g., cyclosporin), and anti-allergic agents (e.g., olopatadine). For example, and without limitation, the therapeutic agent can be apraclonidine or apraclonidine hydrochloride. As a further non-limiting example the therapeutic agent can be olopatadine or olopatadine hydrochloride.

The ophthalmic drug may be present in the form of a pharmaceutically acceptable free base, such as apraclonidine free base or olopatadine free base, or a pharmaceutically acceptable free salt, such as apraclonidine hydrochloride or olopatadine hydrochloride. The therapeutic agent can be anionic, cationic, or neutral. In the event the therapeutic agent selected is anionic in an aqueous solution at an ophthalmically acceptable pH level, buffers described herein are be included.

The therapeutic agent has a concentration of 0.01% w/v to 1% w/v. For example, the therapeutic agent has a concentration of about 0.06% w/v to about 0.8% w/v, e.g., about 0.06% w/v, about 0.12 to about 0.13% w/v, or about 0.77% w/v to about 0.78% w/v, or the like. As a further non-limiting example, the therapeutic agent can be apraclonidine, in which the concentration is from 0.06% w/v to 0.125% w/v, e.g., 0.06% w/v, or 0.125% w/v. As a further non-limiting example, the therapeutic agent can be olopatadine, and the concentration is from 0.6% w/v to 0.8% w/v, e.g., 0.7% w/v.

The present disclosure is particularly directed to ophthalmic compositions in connection with the treatment of conditions wherein the cornea or adjacent ocular tissues are irritated, or conditions requiring frequent application of a composition, such as in the treatment of dry eye patients, red eye patients, or patients that have an ocular allergy. The ophthalmic compositions of the present disclosure are therefore particularly useful in the field of artificial tears, ocular lubricants, and other compositions used to treat dry eye conditions, as well as other conditions involving ocular inflammation or discomfort.

The ophthalmic compositions of the present disclosure include one or more viscosifying agents to enhance bioavailability of the therapeutic agent in the ophthalmic composition. Additionally, the viscosifying agent can provide ocular comfort and/or retention of the compositions on the eye following topical application. The types of viscosifying agents which may be utilized include: water soluble cellulose derivatives, such as cellulose ethers, such as hydroxypropyl guar referred to hereinafter as (“hp-guar”), hydroxypropyl methylcellulose (“HPMC”): Dextran 70, hydroxy methylcellulose (“HMC”), hydroxy ethylcellulose (“HEC”), or hydroxy propyl cellulose (“HPC”); polyethylene glycol; polyethylene oxide polymers; polyvinylpyrrolidone polymers, such as N-vinyl-2-pyrrolidone; propylene glycol; carboxy vinyl polymers; water soluble polyvinyl alcohol polymers or copolymers; copolymers having at least one vinyl lactam with one or more hydrophilic monomors, and polysaccharides. For example, and without limitation, the viscosifying agent can be hydroxypropyl methylcellulose, which provides enhanced bioavailability of the therapeutic agent compared to other viscosifying agents.

The viscosifying agent can include one or more copolymers of vinylpyrrolidone having one or more hydrophilic monomeric units. The viscosifying agent may include polyvinylpyrrolidone copolymers having a copolymer of vinylpyrrolidone and at least one amino containing vinylic monomer. An amino containing vinylic monomer can include alkylaminoalkylmethacrylate having 8-15 carbon atoms, alkylaminoalkylacrylate having 7-15 carbon atoms, dialkylaminoalkylmethacrylate having 8-20 carbon atoms, dialkylaminoalkylacrylate having 7-20 carbon atoms, N-vinylalkylamide having 3-10 carbon atoms. A copolymer of vinylpyrrolidone can be N-vinyl alkylamide, such as N-vinyl formaide, N-vinyl acetamide, N-vinyl isopropylamide, N-vinyl-N-methyl acetamide, or the like. For example, and without limitation, the viscosifying agent is N-vinyl-2-pyrrolidone.

The viscosifying agent is present in the composition in an amount of 0.01% w/v to 5% w/v, such as from 0.01% w/v to 3% w/v, and preferably 0.1% to 0.8% w/v.

Ophthalmic compositions of the present disclosure can include a buffering agent. The buffering agent maintains the pH at a physiological acceptable range of about 6 to about 8. The buffering agent can include citric acid, citrates, boric acid, borates, e.g., sodium borate, bicarbonates, e.g., sodium bicarbonate, sodium hydroxide, hydrochloric acid, TRIS (2-amino-2-hydroxymethyl-1,3-propanediol), Bis-Tris (Bis-(2-hydroxyethyl)-imino-tris-(hydroxymethyl) methane), bis-aminopolyols, triethanolamine, ACES (N-(2-hydroxyethyl)-2-aminoethanesulfonic acid), BES (N,N-Bis(2-hydroxyethyl)-2-aminoethanesulfonic acid), HEPES (4-2-hydroxyethyl)-1-piperazineethanesulfonic acid), MES (2-(N-morpholino) ethanesulfonic acid), MOPS (3-[N-morpholino]-propanesulfonic acid), PIPES (piperazine-N,N′-bis(2-ethanesulfonic acid), TES (N-[Tris(hydroxymethyl)methyl]-2-aminoethanesulfonic acid), phosphate buffers, e.g., NaHPO₄, NaH₂PO₄, NaH₂PO₄, and KH₂PO₄ or hydrates or mixtures thereof, or combinations of one or more buffer agent. For example, and without limitation, an ophthalmic composition may include a mixture of buffering agents including boric acid, sodium hydroxide, and hydrochloric acid. As a further non-limiting example, a buffering agent may include a mixture of buffering agents including sodium chloride, sodium citrate dehydrate, sodium hydroxide, and hydrochloric acid.

The buffering agent can have a concentration in the ophthalmic composition to maintain a pH of the composition of about 6.0 to about 8.5. The concentration of each buffering agent may be 0.001% w/v to 2% w/v. For example, and without limitation, each buffering agent can have a concentration of 0.01% w/v to 1% w/v, such as 0.05% w/v to 0.30% w/v.

The ophthalmic composition of the present disclosure can be isotonic with a lacrimal fluid. A solution which is isotonic with a lacrimal fluid is generally understood to be a solution whose concentration corresponds to the concentration of 0.7% w/v to 1.5% w/v of sodium chloride solution (308 mOsm/kg), e.g., about 0.9% w/v. The tonicity of the ophthalmic composition can be adjusted by adding one or more tonicity agents, e.g., organic or inorganic substances, which affect the tonicity. Tonicity agents can include sodium chloride, potassium chloride, glycerol, propylene glycol, polyols, mannitol, sorbitol, xylitol and mixtures thereof. The tonicity of the solution is typically adjusted to be in the range from 200 to 450 milliosmoles per kilogram (mOsm/kg), preferably from 210 to 350 mOsm/kg.

An ophthalmic composition of the present disclosure includes a surfactant, which may be a non-ionic, anionic, or amphoteric surfactant. In one embodiment, the surfactant is a non-ionic, anionic, or amphoteric surfactant having at least one polyether alcohol moiety. Suitable surfactants include alkyl aryl polyether alcohols, e.g., tyloxapol, poloxamers, e.g., Pluronic® F108, F88, F68, F68LF, F127, F87, F77, P85, P75, P104, and P84, poloxamines, e.g., Tetronic 707, 1107 and 1307, polyethyleneglycol esters of fatty acids, e.g., Tween® 20 or Tween® 80, polyoxyethylene or polyoxypropylene ethers of C₁₂-C₁₈ alkanes, e.g., polyethylene glycol 400 or Brij® 35, polyoxyethyene stearates, e.g., Myrj® 52 or polyoxyl 40 stearate, sorbitol, sorbitan esters e.g., sorbitan monostearate, sorbitan tristearte, or sorbitan monolaurate, polyoxyethylene propylene glycol stearates, e.g., Atlas G2612, amphoteric surfactants under the trade names Mirataine® and Miranol®, or the like. The surfactant may include a combination of one or more surfactants. Preferred surfactants are tyloxapol, polysorbate 80, and polyoxyl 40 stearate.

The surfactant can be present in the ophthalmic composition at a concentration of 7% w/v or less, e.g., 0.005% w/v to 7% w/v, or 0.01% w/v to 2% w/v, or 0.01% w/v to 1% w/v, or 0.01 to 0.5% w/v. As a non-limiting example, a surfactant including tyloxapol may have a concentration of 0.05% w/v to 0.3% w/v. As a further non-limiting example, a surfactant including polysorbate 80 may have a concentration of 0.1% w/v to 0.2% w/v. As a further non-limiting example, a surfactant including polyoxyl 40 stearate may have a concentration of 0.1% w/v to 7% w/v. The surfactant in the ophthalmic composition allows for enhanced control of the dosing and promotes a stable drop size after each successive dose. Additionally, the surfactant allows for less overall ophthalmic composition needed to provide sufficient dosing amounts of therapeutic agent. Additionally, or alternatively, and without wishing to be bound by theory a surfactant comprising a polyether alcohol moiety can cause drop size to be reduced due to the interaction of the alcohol moiety with the surrounding diluents, in which a surface tension of the composition is reduced.

A surface tension of the ophthalmic composition may decrease as the surfactant concentration increases. The surface tension of the ophthalmic composition can be about 30 mN/m to about 45 mN/m, e.g., about 30 mN/m to about 40 mN/m, about 35 mN/m to about 40 mN/m, about 37 mN/m to about 39 mN/m, or the like. Without wishing to be bound by theory, a lower surface tension may cause the drop size to be reduced in the ophthalmic composition, in which tyloxapol, polyoxyl 40 stearate, and polysorbate 80 each reduce the surface tension of the ophthalmic compositions described herein. For example, as the surfactant concentration increases from a first surfactant concentration to a second surfactant concentration, in which the second surfactant concentration is greater than the first surfactant concentration, a surface tension of the ophthalmic concentration may decrease from a first surface tension to a second surface tension, in which the second surface tension is lower than the first surface tension.

The ophthalmic composition of the present disclosure is generally formulated as a sterile solution having one or more diluents, such as water, to form a sterile aqueous solution. The diluents can include any suitable diluent capable of acting as an ophthalmic composition, in which a suitable aqueous solvent is one that is compatible with an eye and/or other tissues to be treated with the ophthalmic composition. For example, and without limitation, the diluent of the ophthalmic composition described herein can be water.

The compositions of the present disclosure are formulated so as to be compatible with the eye and/or other tissues to be treated with the ophthalmic compositions. The ophthalmic compositions intended for direct application to the eye will be formulated so as to have a pH and tonicity which are compatible with the eye. Importantly, the ophthalmic composition of the present disclosure lacks a preservative, which can cause irritation to the eye and/or other tissues to be treated with the ophthalmic compositions described herein.

### Multidose Preservative-Free (MDPF) System

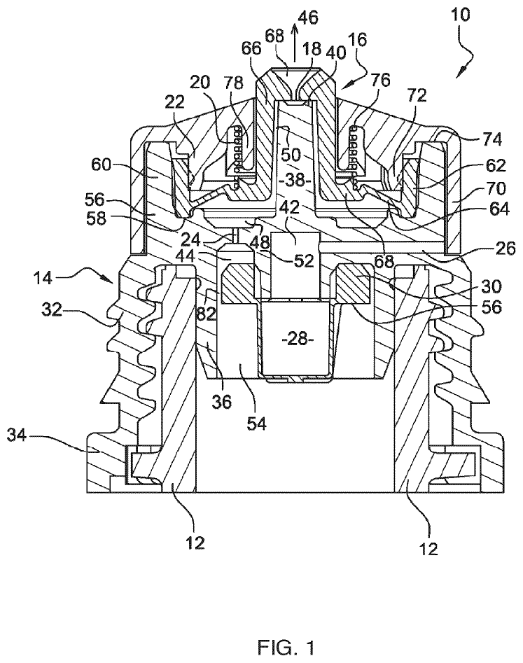

FIG. 1 shows an end piece 10 of a multidose preservative-free eye dropper bottle system for dispensing liquid in the form of a drop, for screw mounting onto the neck of a reservoir 12. This reservoir 12 is a storage reservoir for a liquid, for example the ophthalmic composition described herein. The reservoir 12 includes a volume of about 1 mL to about 15 mL, e.g., about 5 mL, about 7 mL, about 8 mL, about 11 mL, about 15 mL or the like. The reservoir 12 can be deformed so as to dispense liquid by pressing on the reservoir. The liquid is dispensed by pressure, which can be applied by a user, on the body of the reservoir 12. The reservoir has an elasticity to enable it to return to its initial shape after the pressure exerted by the user is released, generating a depression inside the reservoir 12.

In this example, the end piece 10 includes a support 14, a dispensing valve 16 equipped with a dispensing opening 18, a spring 20, an outer envelope 22, a channel 24 for the passage of liquid from the reservoir 12 to the dispensing opening 18 and a channel 26 for the passage of air into the reservoir 12, channel 26 being closed off by an air-permeable member 28.

In this example, the support 14 includes a fastening part 32 for fastening to the reservoir 12, placed at the proximal end of the support 14. The fastening part 32 comprises an external skirt 34 including a screw thread enabling it to be screwed onto the neck of the reservoir 12. The fastening part 32 also includes a tubular internal skirt 36, enabling it to ensure the seal between the reservoir 12 and the dispensing end piece 10.

Furthermore, the support 14 includes a central sealing part 38, cylindrical in shape and extending in the distal direction, opposite to the internal skirt 36. The central sealing part 38 comprises, on its distal end, a bearing surface 40 of the valve 16 to block the flow of liquid in blocking configuration. In this example, the bearing surface 40 has an annular bead shape.

In this example, the support 14 also comprises the channel 26 for the passage of air into the reservoir 12, which opens to a proximal cylindrical cavity 42. This cavity 42 opens, at its proximal end, to the member 28.

In this example, the support 14 also comprises a housing 44 forming a cylindrical cavity, this cavity opening to the reservoir 12 at its proximal end and opening to the channel 24 for the passage of liquid at its distal end, formed in the support 14 and extending in the longitudinal direction of the device, corresponding in this case to the direction of liquid ejection illustrated by the arrow 46. Channel 24 opens to an intermediate cavity 48, itself opening to a second channel 50 for the passage of liquid.

The housing 44 is next to the cavity 42, being separated by an annular wall 52, extending in the direction opposite to the sealing part 38.

The air-permeable member 28 is made of an air-permeable polymeric material e.g., polypropylene, this material being non-porous, blocking the passage of particles such as bacteria of diameter about 0.1 micrometers, but allowing molecules, such as air molecules, e.g., about 0.299 nm to about 0.363 nm, to pass. Air passes through the air-permeable member 28 by diffusion across the member 28. The polymeric material comprises an elastomer material, e.g., silicone. The member 28 has a generally cylindrical or conical shape. Its central axis is co-linear with that of the end piece 10, this axis corresponding to the liquid-dispensing direction, therefore to the arrow 46. For example, the member 28 includes an air passage wall, which promotes the exchange of gases, cylindrical or conical in shape, with a top closed off by a disc-shaped surface, and a base comprising an annular collar 30 for fastening on the end piece 10, this collar 30 being thicker than the thickness of the air passage wall.

The member 28 is housed in a bounded cylindrical cavity 54, which is bounded by the internal skirt 36 of the support 14 and is fastened, e.g., by mechanical tightening or by cooperation of the collar 30 with the annular wall 52. The inner diameter of the collar 30 is less than the outer diameter of the wall 52, such that the collar is held against the wall 52 by elasticity. Snap fastening means can include an inner annular bead formed on the collar 30 snap fastening into an annular groove formed on the outer surface on the wall 52. Additionally, mechanical means for fastening the collar 30 on the wall 52 can be utilized. Mechanical attachment means crossing the part 14 to reach the cavity 48 or means for attaching onto the inner wall of the cylinder are also envisioned in the present disclosure.

In addition, the support 14 comprises a part 56 for fastening the valve 16 on the support 14. This part 56 also acts as part used for fastening the outer envelope 22 on the support 14. It comprises an annular groove 58 bounded on the periphery by an annular wall 60. The annular groove 58 is also bounded, on its inner periphery, by an annular rib created on a wall, forming a disc, crossed by the channel 24 and bounding the cavity 48

The valve 16 can be include an elastomer material, in which the valve 16 can take a configuration for blocking liquid and a configuration for dispensing liquid, by cooperation with the support 14. Alternatively, only part of the valve 16 may be made from an elastomer material, the other part being made from a more rigid material, which can act as seat for the spring 20. The valve 16 includes a fastening part 62 for fastening to the support 14, forming a tubular skirt. This fastening part 62 is connected to a disc-shaped web 64 and from which a cylindrical central part 66 projects out. The web 64 also comprises a seat 68 for the spring 20. The part 66 forms a cylindrical inner cavity, complementary to the part 38. The part 38 and the cylindrical part 66 are coaxial and jointly bound the channel 50 for the passage of liquid. This channel 50 for the passage of liquid opens to the dispensing opening 18 formed in the distal end of the valve 16, itself opening to a shape for forming drops.

The outer envelope 22 includes an annular part 70 for fastening on the support 14, as well as the annular part 72, coaxial with the annular part 70, so as to form a groove 74 housing the annular wall 60. The outer envelope 22 also includes a seat 76 for the spring 20, extended on its inner periphery by an annular wall 78, crossed by the part 66 and designed to center the part 66 of the valve 16.

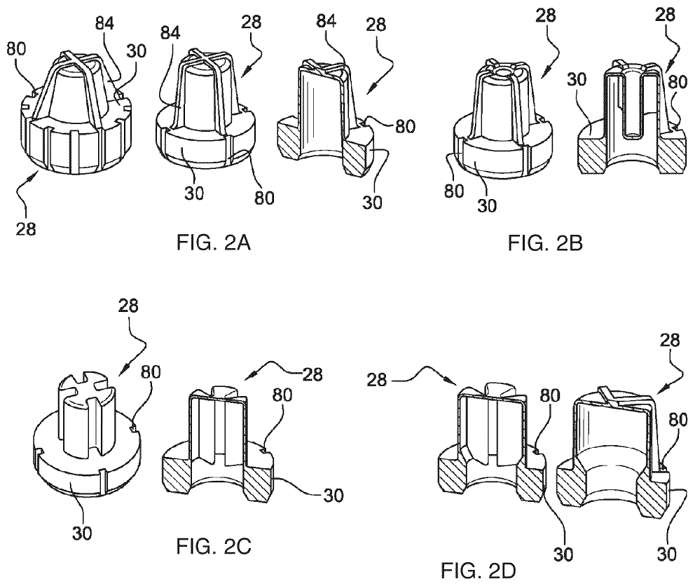

The air-permeable member 28 includes at least one channel 80 for the passage of liquid. The channel 80 for the passage of liquid acts as flow limiter for the liquid, opening to the channel 24 for the passage of liquid. The collar 30 of the member 28 comprises on its outer annular surface a plurality of grooves 80, as shown in FIGS. 2A-2D, and delimiting channels 82, for reducing the flow of liquid. These delimiting channels 82 have a relatively small diameter to reduce the liquid pressure when the user presses on the reservoir. The grooves 80 can have changes of direction or a spiral shape. Depending on the number and size of the grooves 80 placed opposite the housing 44, the flow of liquid coming out will be more or less reduced.

For example, the member 28 can take one of the shapes illustrated on FIGS. 2A-2D. The reduction shapes 80 are made on the outer periphery of its collar 30, forming a recess in the periphery.

The member 28 includes a thin air passage wall, of cylindrical or conical shape. To make it more rigid, the wall also comprises stiffening ribs 84, corresponding to local increases in the thickness of the wall, as shown in FIG. 2A.

The members 28 of FIGS. 2B-2D illustrate other types of members 28 on which the air passage wall includes a plurality of reliefs which increase the air exchange area between the inside and outside of the reservoir 12 without making the member 28 larger. These reliefs are formed in the wall so that it remains relatively thin to allow air to pass. In addition, these reliefs can be used to make the member 28 more rigid, avoiding the need for the stiffeners 84, as shown in particular on FIG. 2C, which illustrates a corrugated air passage wall, having a clover-shaped cross-section.

Referring again to FIG. 1, at rest, e.g., when no user is pressing the reservoir 12, the valve 16 is in configuration for blocking liquid, e.g., it presses on the surface 40, since it is permanently fastened to the Support 14, exerting an elastic force on the valve, and due to the pressure exerted by the spring 20.

A force is applied to the reservoir 12 exerting a pressure on the fluid which flows into the only channel allowing it to flow, e.g., the channel 82 for passage of liquid. Without being bound by theory, a multi-dose pharmaceutical composition having a surfactant can reduce the amount of actuation force or pressure needed to dispense a dose. As it passes through this channel 82 the fluid flow rate decreases, due to the pressure drop. The fluid flows in the channel 24, to the cavity 48 and then to the channel 50. Under the effect of the pressure, the fluid lifts the valve 16, which then switches into configuration for dispensing liquid, and can therefore flow between the valve 16 and the bearing surface 40, to pass in the channel and in the cavity, and therefore take the form of a drop.

Once the drop has been dispensed, the user releases the pressure on the deformable reservoir 12, which tends to take up its initial shape, generating a depression inside the reservoir 12. This depression will be compensated by an intake of exterior air from the channel 26 for the passage of air through the air-permeable member 28, which may require about 1 second to about 24 hours due to the material forming the member 28 being non-porous. Without being bound by theory, the deformable reservoir 12 may become deformed due to a rapid dispensing of the pharmaceutical composition, in which a rapid dispensing can include dispensing more than 1 drop per second.

### Methods of Forming a Multidose System

The present disclosure provides a method of forming a multidose system. The method includes receiving an ophthalmic composition having a therapeutic agent, a viscosifying agent having a concentration of 0.01% w/v to 0.5% w/v, and a surfactant having a concentration of 0.005% w/v to 7% w/v, but lacking a preservative. The method includes dispensing an amount of the ophthalmic composition into a reservoir of an MDPF dropper bottle system. The ophthlamic composition is heat sterilized and/or sterile filtered. The method can include disposing about 1 mL to about 15 mL, such as about 11 mL, of the ophthalmic composition into the reservoir, e.g., about 5 mL in Class A clean area (room, filling line) under laminar flow. An end piece of the MDPF dropper bottle system is fastened to the reservoir, e.g., by mechanical tightening, by cooperation of a collar 30 with an annular wall 52.

### EXAMPLES

### Example 1: Drop Size of Ophthalmic Composition a with No Surfactant

The compositions shown in Table 1 below were prepared as follows.

TABLE 1

| | Compositions (Concentration, % W/V) | | | | |
|---|---|---|---|---|---|
| Ingredients | A1 | A2 | A3 | A4 | A5 |
| Apraclonidine | 0.06 | 0.08 | 0.125 | 0.2 | N/A |
| HPMC - | 0.3 | 0.3 | 0.3 | 0.3 | 0.3 |
| Hydroxypropyl | | | | | |
| methylcellulose | | | | | |
| Citric acid | 0.0067 | 0.0067 | 0.0067 | 0.0067 | 0.0067 |
| Sodium citrate | 0.45 | 0.45 | 0.45 | 0.45 | 0.45 |
| Dihydrate | | | | | |
| (Na₃C₆H₅O₇) | | | | | |
| Sodium chloride | 0.74 | 0.74 | 0.74 | 0.74 | 0.74 |
| 1N Sodium | As | As | As | As | As |
| hydroxide | needed | needed | needed | needed | needed |
| (NaOH) | | | | | |
| 1N | As | As | As | As | As |
| Hydrochloric | needed | needed | needed | needed | needed |
| acid (HCl) | | | | | |
| H₂O | QS to | QS to | QS to | QS to | QS to |
| | 100% | 100% | 100% | 100% | 100% |

Composition A1, composition A2, composition A3, composition A4, and composition A5 each had densities of 1.0063 g/mL, 1.0065 g/mL, 1.0065 g/mL, 1.0066 g/mL, and 1.0058 g/mL, respectively.

### Example 2: Drop Size of Ophthalmic Composition A with Tyloxapol, Polysorbate 80, or Polyoxyl 40 Stearate

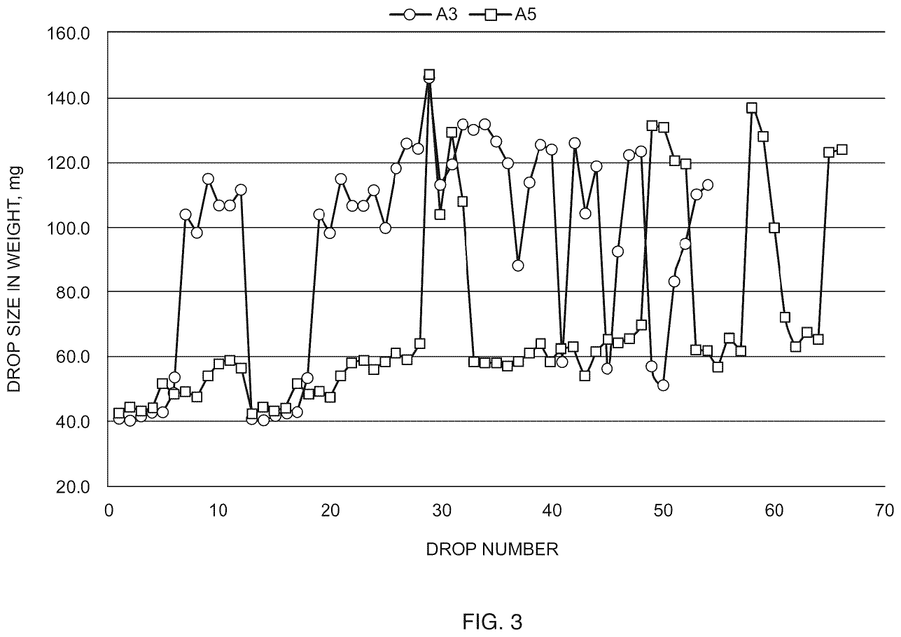

Spreading and drop size increases occurred in both the composition A3 and composition A5 samples, as shown in Table 3, and FIG. 3. A surfactant including tyloxapol, polysorbate 80, or polyoxyl 40 stearate having a composition of 0.100% w/v, 0.025% w/v, or 0.010% w/v was added to composition A3 and composition A5, shown above in Tables 4-6. Drop sizes of the compositions having 0.10% w/v, 0.025% w/v, and 0.010% w/v of tyloxapol were stable over time, in which no drop size increase occurred. Additionally, a higher concentration of tyloxapol resulted in a smaller drop size, as shown in Table 4. Without being bound by theory, the prevention of drop size increases allows for a consistent dosage of an ophthalmic composition, as well as provides enhanced control over the location the dosage is delivered as larger droplets may be too large for an eye cavity to hold.

TABLE 3

| Sample | A3 | A5 |
|---|---|---|
| Min | 40.3 | 42.4 |
| Max | 146.4 | 147.4 |
| Mean | 99.1 | 74.1 |
| StDev | 31.3 | 29.4 |
| % RSD | 31.6 | 39.7 |

Drop sizes of the compositions having 0.10% w/v, 0.025% w/v, and 0.010% w/v of tyloxapol were stable over time, in which a higher concentration of tyloxapol resulted in a smaller drop size, as shown in Table 4.

TABLE 4

| | Sample | | | | | |
|---|---|---|---|---|---|---|
| | A3 + | A5 + | A3 + | A5 + | A3 + | A5 + |
| | Tyloxapol | Tyloxapol | Tyloxapol | Tyloxapol | Tyloxapol | Tyloxapol |
| | 0.10% | 0.10% | 0.025% | 0.025% | 0.01% | 0.01% |
| Min | 25.7 | 25.7 | 32.0 | 30.4 | 35.5 | 34.4 |
| Max | 40.2 | 53.4 | 46.4 | 51.9 | 49.1 | 64.5 |
| Mean | 29.9 | 31.8 | 41.0 | 41.3 | 43.3 | 45.0 |
| StDev | 3.1 | 5.9 | 2.6 | 3.3 | 2.7 | 4.3 |
| % RSD | 10.5 | 18.6 | 6.4 | 8.1 | 6.2 | 9.6 |

Drop sizes of the compositions having 0.10% w/v, 0.025% w/v, and 0.010% w/v of polysorbate 80 were stable over time, in which a higher concentration of polysorbate resulted in a smaller drop size, as shown in Table 5.

TABLE 5

| | Sample | | | | | |
|---|---|---|---|---|---|---|
| | A3 + | A5 + | A3 + | A5 + | A3 + | A5 + |
| | Polysorbate | Polysorbate | Polysorbate | Polysorbate | Polysorbate | Polysorbate |
| | 80 | 80 | 80 | 80 | 80 | 80 |
| | 0.10% | 0.10% | 0.025% | 0.025% | 0.01% | 0.01% |
| Min | 29.5 | 26.4 | 17.0 | 31.9 | 35.8 | 36.8 |
| Max | 41.4 | 43.5 | 48.5 | 48.3 | 51.8 | 52.2 |
| Mean | 36.4 | 36.8 | 40.4 | 41.5 | 42.9 | 45.5 |
| StDev | 2.5 | 3.0 | 4.0 | 2.5 | 3.0 | 2.9 |
| % RSD | 6.8 | 8.2 | 9.8 | 6.1 | 7.0 | 6.3 |

Drop sizes of the compositions having 0.10% w/v, 0.025% w/v, and 0.010% w/v of polyoxyl 40 stearate were stable over time, in which a higher concentration of polyoxyl 40 stearate resulted in a smaller drop size, as shown in Table 6.

TABLE 6

| | Sample | | | | | |
|---|---|---|---|---|---|---|
| | A3 + | A5 + | A3 + | A5 + | A3 + | A5 + |
| | Polyoxyl | Polyoxyl | Polyoxyl | Polyoxyl | Polyoxyl | Polyoxyl |
| | 40 stearate | 40 stearate | 40 stearate | 40 stearate | 40 stearate | 40 stearate |
| | 0.10% | 0.10% | 0.025% | 0.025% | 0.01% | 0.01% |
| Min | 30.3 | 33.9 | 37.7 | 33.8 | 36.1 | 41.5 |
| Max | 43.2 | 45.5 | 48.4 | 56.0 | 87.1 | 70.0 |
| Mean | 38.8 | 39.7 | 43.1 | 44.6 | 45.3 | 47.4 |
| StDev | 2.8 | 2.3 | 2.1 | 4.2 | 6.0 | 5.0 |
| % RSD | 7.2 | 5.9 | 4.9 | 9.5 | 13.3 | 10.6 |

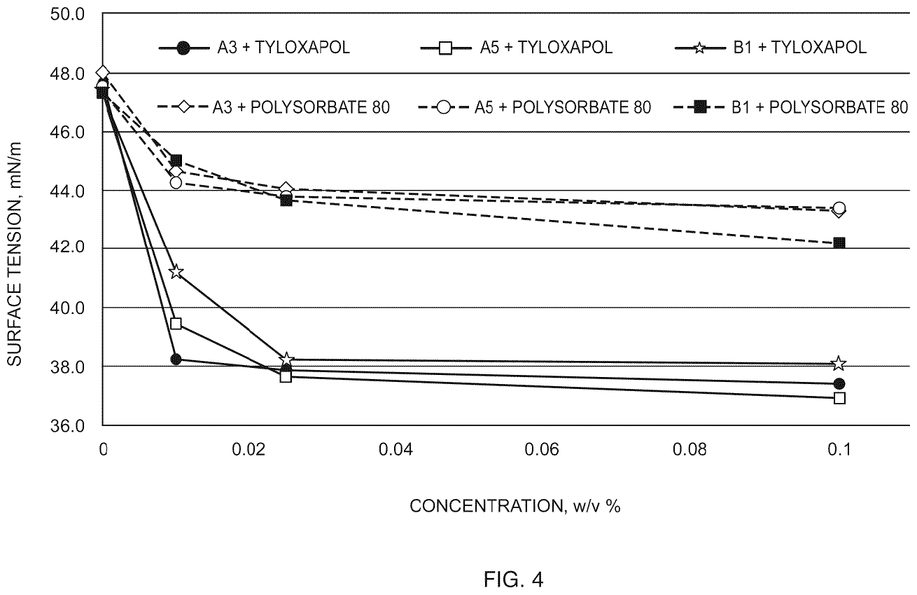

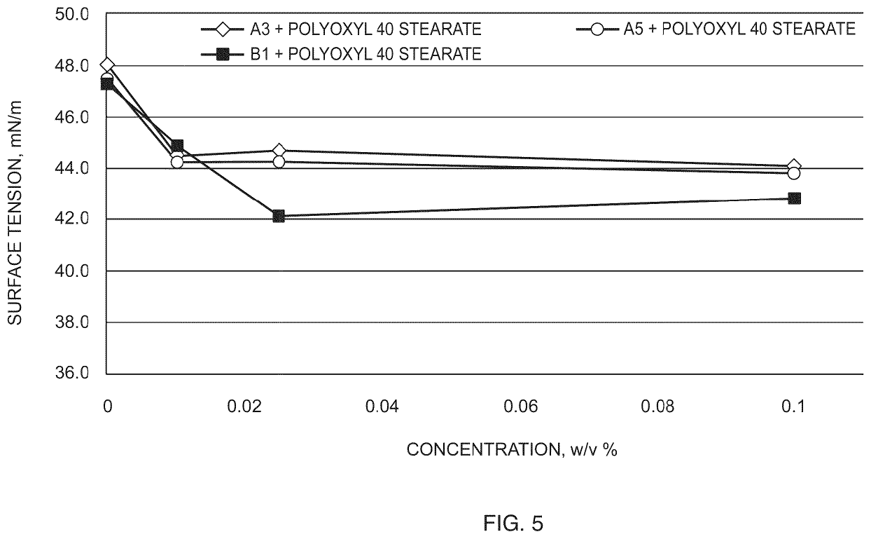

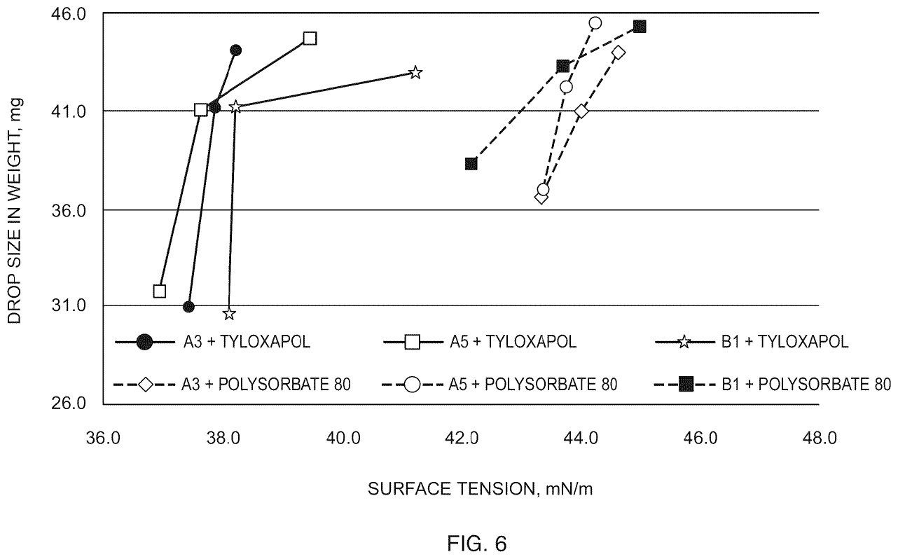

The concentrations of 0.10% w/v for each of the surfactants resulted in a lower corresponding surface tension, as shown in FIGS. 4 and 5. Tyloxapol reduced the surface tension the most, when compared with polyoxyl 40 stearate or polysorbate 80, as shown in FIGS. 4 and 5. Moreover, the concentrations of 0.10% w/v for each of the surfactants resulted in a smaller drop size of the sample, as shown in FIG. 6, which is represented as the smallest drop size in weight. The middle drop size in weight of each respective sample was the 0.025% w/v of surfactant, and the largest drop size in weight of each respective sample was 0.010% w/v of surfactant. Without wishing to be bound by theory, a surfactant having a larger surface activation may cause the drop size to be reduced in the ophthalmic composition, in which tyloxapol has a stronger surface activation, due to the aromatic behavior of the structure, compared to both polyoxyl 40 stearate and polysorbate 80. Without wishing to be bound by theory, a drop size of under 40 μL may be achieved using at least 0.05% w/v tyloxapol, 0.10% w/v polysorbate 80, or 0.10% w/v polyoxyl 40 stearate.

### Example 3: Drop Size of Ophthalmic Composition a with Sorbitol and/or PEG 400

A surfactant including sorbitol and/or PEG 400 having various compositions was added to composition A1. Spreading occurred in all samples, including the samples with sorbitol and/or PEG 400, as shown in Table 7. 0.4% PEG400 in composition A1 reduced the average drop size from 85.7 mg to 79.3 mg, 1.4% Sorbitol increased the drop size, from 85.7 mg to 87.4 mg, as shown in Table 7.

TABLE 7

| | Sample | | | | | |
|---|---|---|---|---|---|---|
| | 2.8% | 1.4% | 0.7% | | | |
| | sorbitol | sorbitol | sorbitol | | | |
| | and 0.8% | and 0.4% | and 0.2% | 1.4% | 0.4% | |
| | PEG400 | PEG400 | PEG400 | sorbitol | PEG400 | |
| | in A1 | in A1 | in A1 | in A1 | in A1 | A1 |
| Min | 41.4 | 42.1 | 42.4 | 43.9 | 41.3 | 42.2 |
| Max | 122.1 | 154.1 | 148.5 | 139.1 | 114.8 | 136.6 |
| Mean | 76.1 | 86.8 | 86.4 | 87.4 | 79.3 | 85.7 |
| StDev | 26.8 | 35.1 | 35.6 | 33.1 | 27.4 | 35.6 |
| % RSD | 35.3 | 40.5 | 41.3 | 37.8 | 34.6 | 41.6 |

### Example 4: Drop Size of Ophthalmic Composition B with Tyloxapol, Polysorbate 80, or Polyoxyl 40 Stearate

The composition shown in Table 8 below was prepared as follows.

A first mixture was prepared by adding approximately 800 mL of purified water to a beaker and heated to about 80° C. to about 90° C. Hydroxypropyl-Gamma-Cyclodextrin was added to the solution and stirred, IN NaOH was added at 25 mL per 100 g of providone raw material. The solution was stirred for 85 to 140 minutes. Polyethylene Glycol (400) and providone K29/32 was added followed by boric acid. Mixing was continued and mannitol was added to the mixture. The mixture was then cooled to about 27° C. to about 40° C. Olopatadine, HCl was then added until dissolved. The pH was then adjusted to 7/0+/−0.1 with IN NaOH. The mixture was then sterilized by sterile filtration with 0.22 μm hydrophilic filters.

A second mixture was prepared by adding, to a separate beaker, 8 g of HPMC in 150 mL of solvent heating to about 60° C. with moderate agitation. 350 mL of cold water, about 1° C. to about 24° C. was added and mixed for about 30 minutes. The solution was then autoclaved and cooled with stirring to room temperature with stirring. Polish filtration 10 μm was used to filter the solution.

The first mixture and second mixture were then slowly mixed and the volume was adjusted by the density weight. The mixture had an osmolality of 297 mOsm, a visual test of yellow, and a viscosity of 15.4.

TABLE 8

| | Compositions | |
|---|---|---|
| | (Concentration, % W/V) | |
| Ingredients | B1 | B1′ |
| Olopatadine, HCl | 0.776 | 0.776 |
| Hydroxypropyl-Gamma-Cyclodextrin | 1.5 | 1.5 |
| Providone K29/32 | 4.0 | 4.0 |
| Polyethylene Glycol (400) | 4.0 | 4.0 |
| HPMC (E4M) | 0.4 | 0.4 |
| Boric Acid | 0.3 | 0.3 |
| Mannitol | 0.2 | 0.2 |
| NaOH, HCl, and H₂O | pH to 7.2, | pH to 7.2, |
| | QS to 100% | QS to 100% |

B1 and B1′ included the same formulation, but were produced at different times, in which B1 was produced first and B1′ was formulated second. Tyloxapol was added to a first set of samples of ophthalmic composition B1 and a first set of samples of ophthalmic composition B1′, in which the tyloxapol had a concentration of 0.100% w/v, 0.025% w/v, 0.010% w/v, as shown in table 9.

TABLE 9

| | Sample | | | | | |
|---|---|---|---|---|---|---|
| | B1 with | B1′ with | B1 with | B1′ with | B1 with | B1′ with |
| | 0.10% w/v | 0.10% w/v | 0.025% w/v | 0.025% w/v | 0.01% w/v | 0.01% w/v |
| | Tyloxapol | Tyloxapol | Tyloxapol | Tyloxapol | Tyloxapol | Tyloxapol |
| Min | 24.9 | 26.3 | 18.8 | 30.9 | 33.8 | 36.4 |
| Max | 43.0 | 40.0 | 48.6 | 51.8 | 50.3 | 53.9 |
| Mean | 30.0 | 30.2 | 38.9 | 41.0 | 41.5 | 43.0 |
| StDev | 3.4 | 3.1 | 4.7 | 3.4 | 3.1 | 3.7 |
| % RSD | 11.3 | 10.2 | 12.0 | 8.4 | 7.4 | 8.6 |

Polysorbate 80 was added to a second set of samples of ophthalmic composition B1, and a second set of samples of ophthalmic composition B1′, in which the polysorbate 80 had a concentration of 0.100% w/v, 0.025% w/v, 0.010% w/v, as shown in table 10.

TABLE 10

| | Sample | | | | | |
|---|---|---|---|---|---|---|
| | B1 with | B1′ with | B1 with | B1′ with | B1 with | B1′ with |
| | 0.10% w/v | 0.10% w/v | 0.025% w/v | 0.025% w/v | 0.01% w/v | 0.01% w/v |
| | Polysorbate | Polysorbate | Polysorbate | Polysorbate | Polysorbate | Polysorbate |
| | 80 | 80 | 80 | 80 | 80 | 80 |
| Min | 30.3 | 28.0 | 29.9 | 29.3 | 33.2 | 32.0 |
| Max | 42.5 | 47.0 | 50.2 | 51.3 | 60.6 | 59.2 |
| Mean | 37.6 | 38.6 | 41.4 | 42.8 | 43.6 | 44.5 |
| StDev | 2.5 | 3.0 | 3.5 | 3.8 | 5.7 | 5.5 |
| % RSD | 6.7 | 7.7 | 8.5 | 8.9 | 13.0 | 12.3 |

Polyoxyl 40 stearate was added to a third set of samples of ophthalmic composition B1, and a third set of samples of ophthalmic composition B1′, in which the Polyoxyl 40 stearate had a concentration of 0.100% w/v, 0.025% w/v, 0.010% w/v, as shown in table 11.

TABLE 11

| | Sample | | | | | |
|---|---|---|---|---|---|---|
| | B1 with | B1′ with | B1 with | B1′ with | B1 with | B1′ with |
| | 0.10% w/v | 0.10% w/v | 0.025% w/v | 0.025% w/v | 0.01% w/v | 0.01% w/v |
| | Polyoxyl | Polyoxyl | Polyoxyl | Polyoxyl | Polyoxyl | Polyoxyl |
| | 40 stearate | 40 stearate | 40 stearate | 40 stearate | 40 stearate | 40 stearate |
| Min | 32.6 | 32.1 | 34.8 | 27.8 | 30.6 | 29.6 |
| Max | 46.6 | 47.8 | 99.6 | 53.1 | 56.0 | 64.0 |
| Mean | 39.1 | 39.8 | 44.9 | 43.7 | 42.7 | 45.3 |
| StDev | 2.9 | 3.3 | 12.0 | 4.8 | 4.3 | 6.2 |
| % RSD | 7.3 | 8.2 | 26.7 | 10.9 | 10.1 | 13.6 |

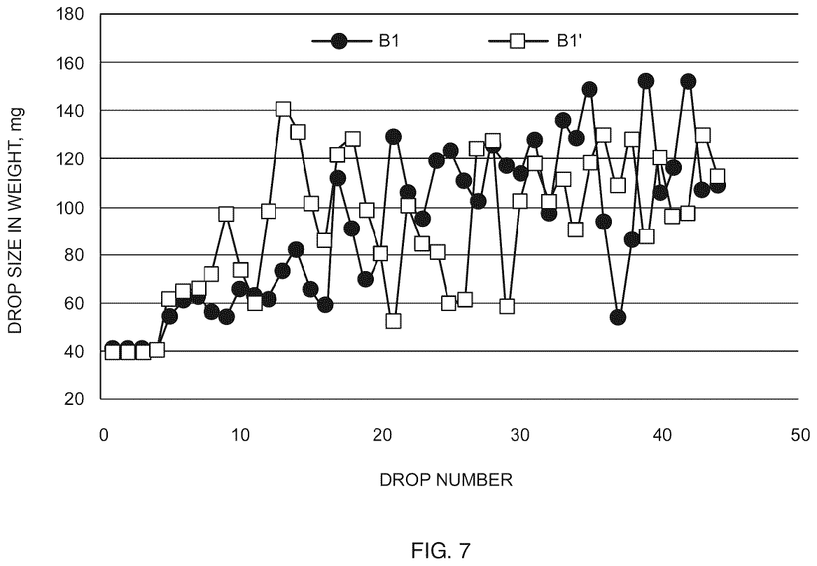

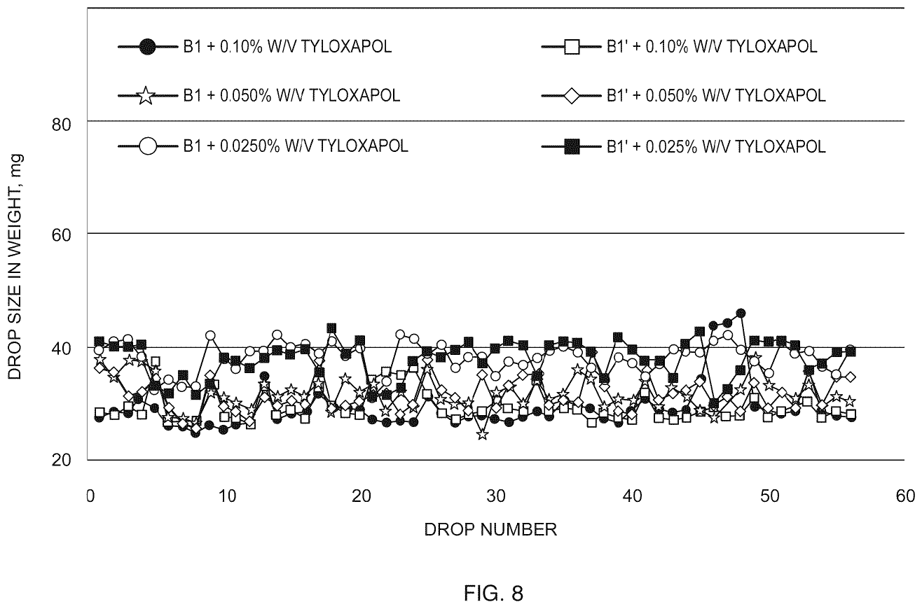

Spreading and drop size increases occurred in composition B1, in which drop size increased from 40 mg to over 100 mg after about 10 drops, as shown in FIG. 7. Drop sizes of the compositions having 0.100% w/v, 0.050% w/v, or 0.025% w/v of tyloxapol were stable over time, in which drop size remained stable between about 40 mg and about 20 mg after more than 50 drops were dispensed, as shown in FIG. 8. Additionally, surface tension was lowered with the addition of 0.1% w/v tyloxapol, as measured by a Biolin Sigma 700/701 tensiometer, described below. Results are shown in FIGS. 4 and 6.

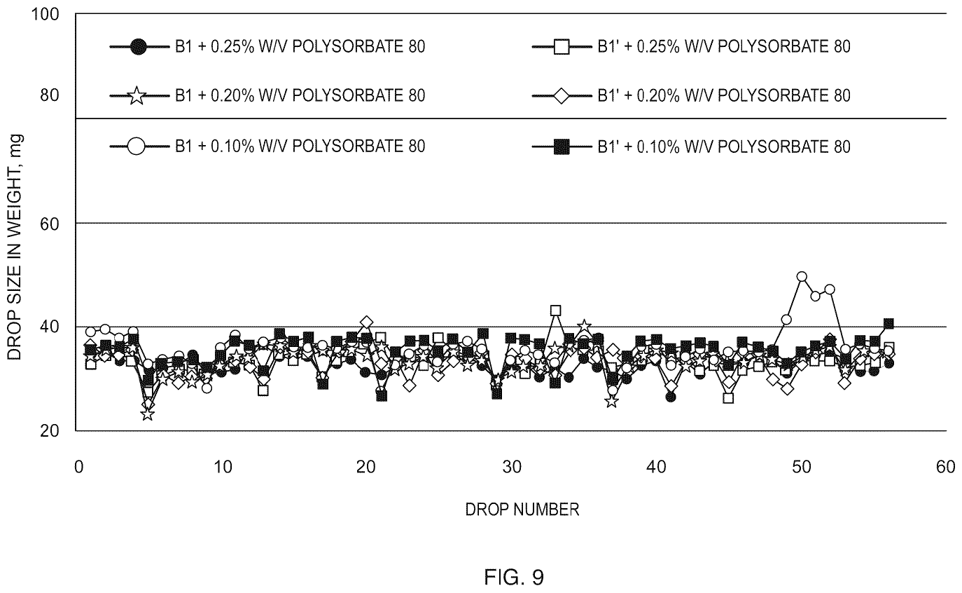

Drop sizes of the compositions having 0.250% w/v and 0.200% w/v, of polysorbate 80 remained stable after more than 50 drops were dispensed, as shown in FIG. 9. Drop sizes of the compositions having 0.100% w/v, of polysorbate 80 exhibited brief spreading after about 50 drops were dispensed, in which the drop size reduced back to below 40 mg after about 52 drops, as shown in FIG. 9. Additionally, surface tension was lowered with the addition of 0.1% w/v polysorbate 80, as shown in FIGS. 4 and 6.

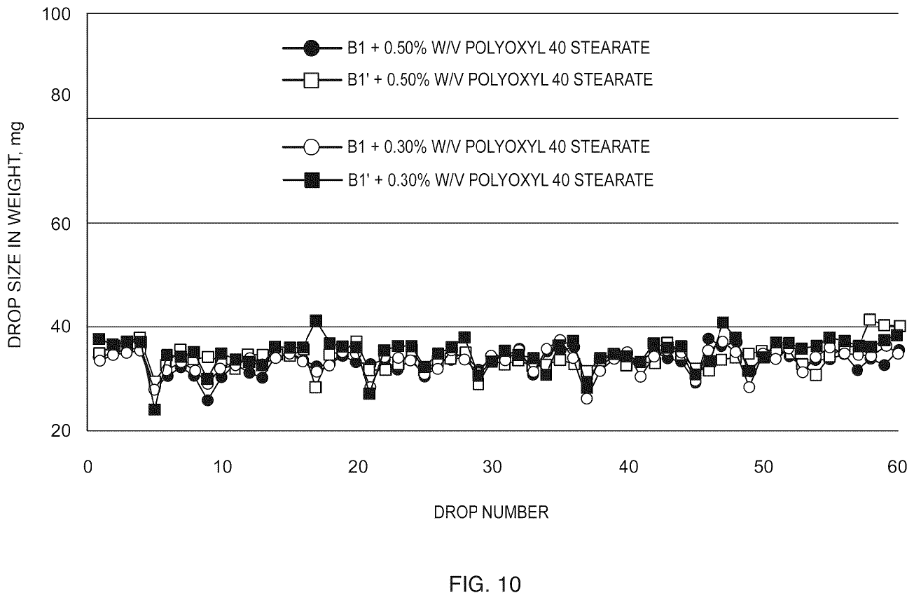

Drop sizes of the compositions having 0.500% w/v and 0.300% w/v, of polyoxyl 40 stearate remained stable after more than 50 drops were dispensed, as shown in FIG. 10. Additionally, surface tension was lowered with the addition of 0.1% w/v polyoxyl 40 stearate, as shown in FIG. 5.

### Example 5: Drop Size of Ophthalmic Composition a with and without Preservatives

Compositions A6-A12 were prepared, in which varying concentrations of therapeutic agent, viscosifying agent, and preservative were added. The variations are presented below in Table 12.

TABLE 12

| | Compositions (Concentration, % W/V) | | | | | | |
|---|---|---|---|---|---|---|---|
| Ingredients | A6 | A7 | A8 | A9 | A10 | A11 | A12 |
| Apraclonidine | 0.08 | 0.08 | — | 0.08 | 0.08 | 0.08 | 0.08 |
| Dihydrogen | 0.093 | 0.093 | 0.093 | 0.092 | 0.092 | 0.092 | 0.092 |
| phosphate | | | | | | | |
| monohydrate | | | | | | | |
| Disodium hydrogen | 0.36 | 0.36 | 0.36 | 0.355 | 0.355 | 0.355 | 0.355 |
| phosphate | | | | | | | |
| heptahydrate | | | | | | | |
| Sodium Chloride | 0.6 | 0.6 | 0.6 | 0.767 | 0.767 | 0.767 | 0.767 |
| Glycerol | 0.45 | 0.45 | 0.45 | — | — | — | — |
| HPMC - | 0.3 | — | — | — | — | 0.484 | 0.484 |
| Hydroxypropyl | | | | | | | |
| methylcellulose | | | | | | | |
| BAK - | 0.01 | 0.01 | 0.01 | — | 0.01 | — | 0.01 |
| Benzyalkykonium | | | | | | | |
| chloride | | | | | | | |
| H₂O | QS to | QS to | QS to | QS to | QS to | QS to | QS to |
| | 100% | 100% | 100% | 100% | 100% | 100% | 100% |

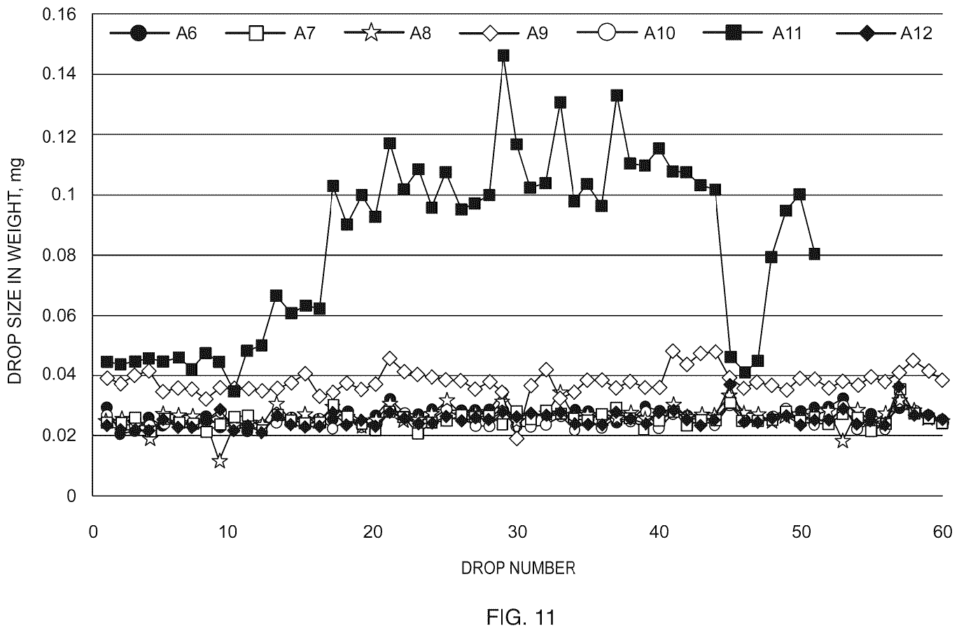

The drop sizes of compositions A6-A8, A10, and A12 remained stable at around 26 mg per drop dispensed, as shown in FIG. 11. The drop size of composition A9, which contained no BAK and no HPMC had a larger drop size than compositions A6-A8, A10, and A12, being about 38 mg, as shown in FIG. 11. The drop size of composition A11, which contained no BAK but HPMC, increased from about 45 mg to about 100 mg over 5 days (about 20 drops), as shown in FIG. 11.

### Example 6: Drop Size of Ophthalmic Composition B with and without Flicking

The composition shown in Table 13 below was prepared as follows.

TABLE 13

| | Compositions (Concentration, % W/V) | | | |
|---|---|---|---|---|
| Ingredients | B2 | B3 | B4 | B5 |
| Olopatadine, | — | 0.12 | — | — |
| HCl | | | | |
| Sodium | — | — | 0.45 | — |
| acetate | | | | |
| Sodium | — | — | 0.75 | — |
| chloride | | | | |
| HPMC | — | — | — | 1.0 |
| H₂O | QS to 100% | QS to 100% | QS to 100% | QS to 100% |

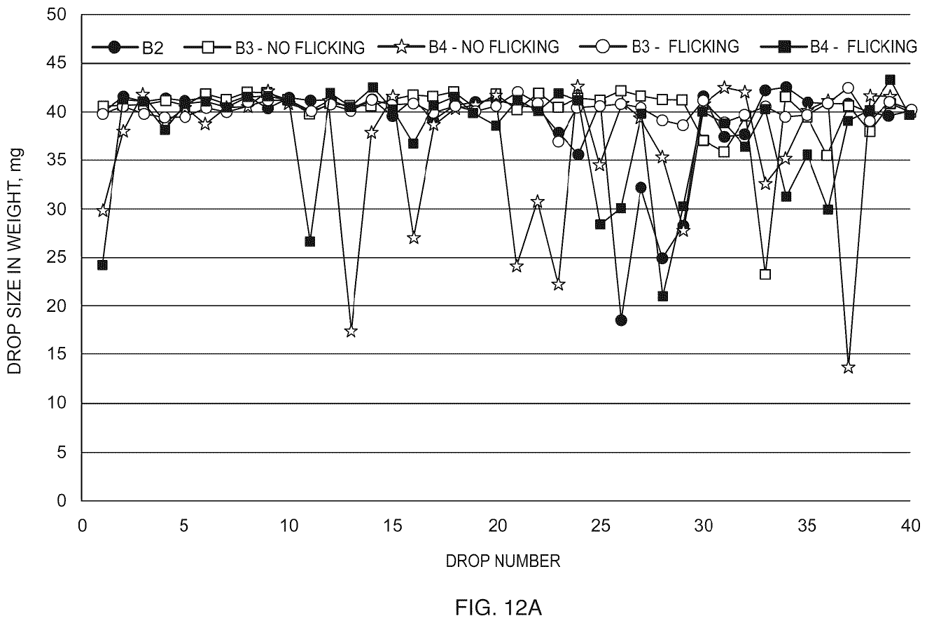

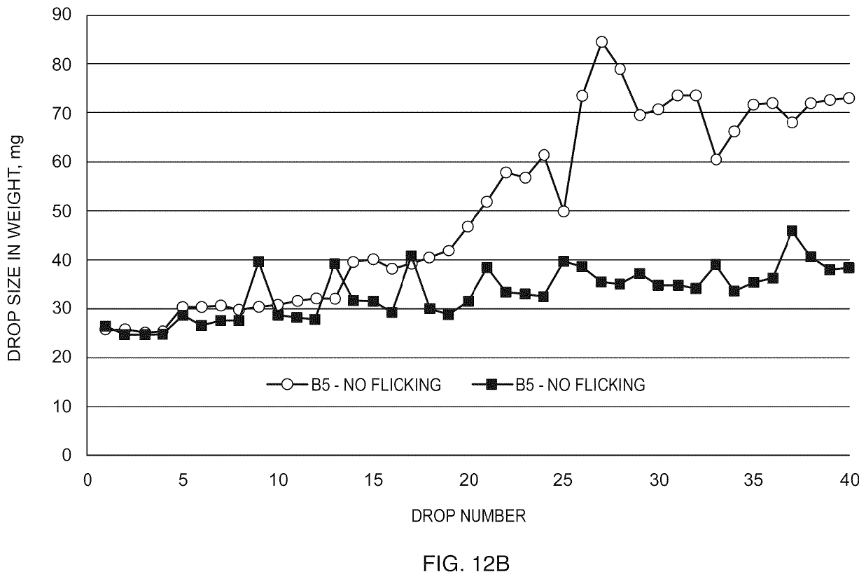

The drop sizes of compositions B2-B4 were about 40 mg, in which smaller drop sizes were observed, as shown in FIG. 12A. Flicking was performed by performing a sudden sharp movement to the end piece of the multidose system described herein. Flicking did not contribute to a change in drop size in compositions B2-B4. The drop sizes of composition B5 with flicking increased from about 25 mg to about 41 mg in 9 days, whereas the drop sizes of composition B5 increased from 25 mg to about 72 mg in 7 days, as shown in FIG. 12B.

### Example 7: Drop Size of Ophthalmic Composition a with Surfactants and/or Preservative

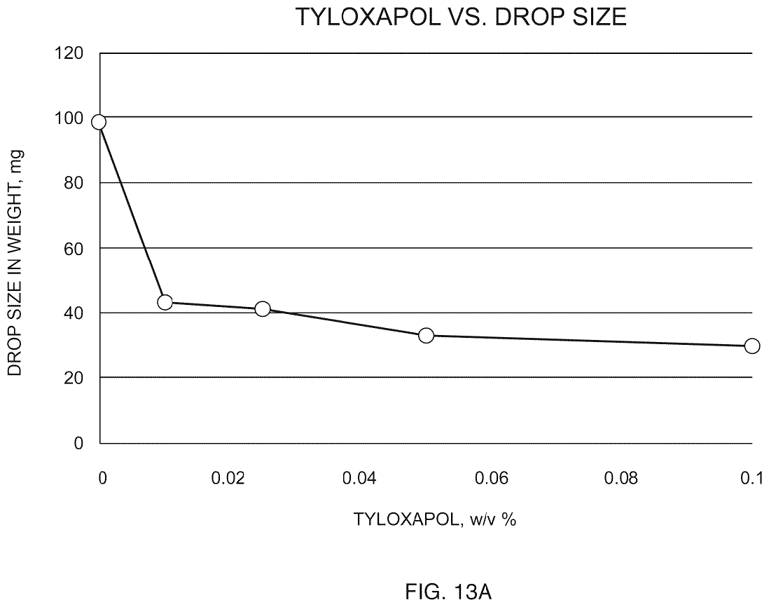

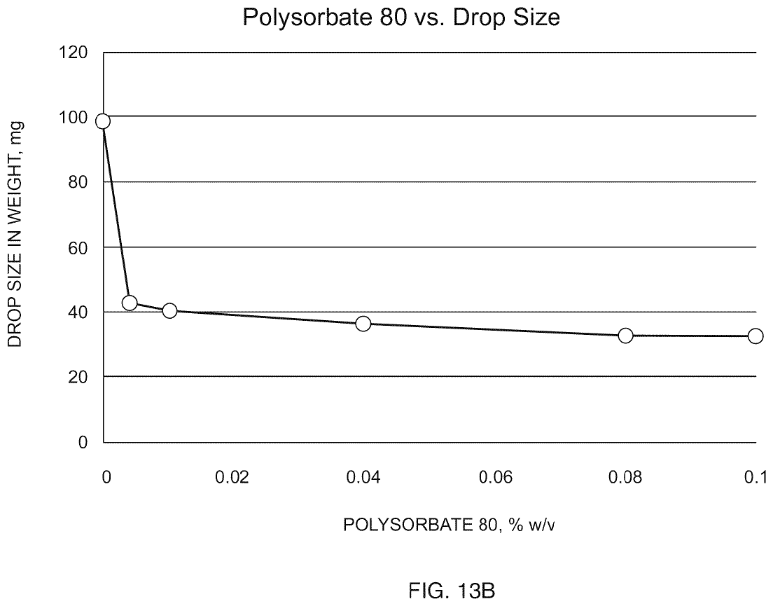

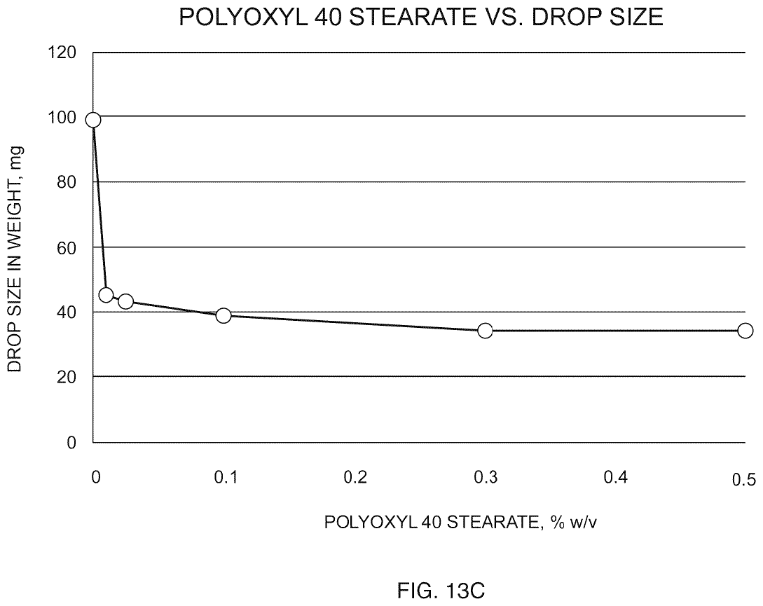

A surfactant including tyloxapol, polysorbate 80, or polyoxyl 40 stearate or a preservative including BAK having a composition of 0.100% w/v, 0.050% w/v, or 0.010% w/v was added to composition A3 shown above in Table 1. Spreading and drop size increases occurred in composition A3, as shown in Table 1, and FIG. 13. Drop sizes of the compositions having 0.050% w/v tyloxapol, 0.100% w/v polysorbate 80, 0.100% polyoxyl 40 stearate, and 0.010% w/v BAK resulted in no spreading or drop size increase, as shown in Table 14.

TABLE 14

| | | A3 + | A3 + | | A3 + |
|---|---|---|---|---|---|
| | | 0.10% | 0.10% | A3 + | 0.01% |
| | | Polyoxyl 40 | Polysorbate | 0.05% | (or 100 |
| Sample | A3 | stearate | 80 | Tyloxapol | ppm) BAK |
| Min | 40.3 | 30.3 | 29.5 | 23.7 | 21.2 |
| Max | 146.4 | 43.2 | 41.4 | 43.3 | 37.0 |
| Mean | 99.1 μL | 38.8 μL | 36.4 μL | 32.8 μL | 26.7 μL |
| StDev | 31.3 | 2.8 | 2.5 | 3.7 | 1.9 |
| % RSD | 31.6 | 7.2 | 6.8 | 11.3 | 7.2 |

Drop size was found to be inversely proportional to surfactant concentration, in which the surfactant concentration that resulted in a drop size of less than 40 mg included 0.05% w/v tyloxapol or 0.1% w/v polyoxyl 40 stearate or 0.1% w/v polysorbate 80, as shown in FIGS. 13A, 13B, and 13C.

### Additional Embodiments

The present disclosure provides, among others, the following aspects, each of which may be considered as optionally including any alternate aspects.
E1. A multidose system for topically administering a composition to the eye comprising:
   a multidose preservative-free dropper bottle system; and
   an ophthalmic composition comprising:
      a therapeutic agent,
      a viscosifying agent comprising a concentration of about 0.01% w/v to about 5% w/v; and
      a surfactant comprising a concentration of about 0.005% w/v to about 7% w/v, wherein the composition lacks a preservative.
E2. The multidose system of embodiment E1, wherein the therapeutic agent is apraclonidine or olopatadine.
E3. The multidose system of embodiment E2, wherein the therapeutic agent is apraclonidine.
E4. The multidose system of embodiment E2, wherein the therapeutic agent is olopatadine.
E5. The multidose system of any one of embodiments E1-E4, wherein the therapeutic agent is present in the composition at a concentration of about 0.01% w/v to about 1% w/v.
E6. The multidose system of embodiment E5, wherein the therapeutic agent is present at a concentration of about 0.01% w/v to about 0.7% w/v.
E7. The multidose system of embodiment E6, wherein the therapeutic agent is present at a concentration of about 0.125% w/v.
E8. The multidose system of embodiment E5, wherein the therapeutic agent is present at a concentration of about 0.70% w/v.
E9. The multidose system of any one of embodiments E1-E8, wherein the viscosifying agent is present at a concentration of about 0.1% w/v to about 0.8% w/v.
E10. The multidose system of embodiment E9, wherein the viscosifying agent is present at a concentration of about 0.3% w/v.
E11. The multidose system of embodiment E9, wherein the viscosifying agent is present at a concentration of about 0.4% w/v.
E12. The multidose system of embodiment E9, wherein the viscosifying agent is hydroxypropyl methylcellulose (HPMC).
E13. The multidose system of any one of embodiments E1-E12, wherein the surfactant is selected from the group consisting of tyloxapol, polysorbate 80, and polyoxyl 40 stearate.
E14. The multidose system of embodiment E12, wherein the surfactant is tyloxapol.
E15. The multidose system of embodiment E12, wherein the surfactant is polysorbate 80.
E16. The multidose system of embodiment E12, wherein the surfactant is polyoxyl 40 stearate.
E17. The multidose system of any one of embodiments E1-E16, wherein the surfactant is present at a concentration of about 0.01% w/v to about 1% w/v.
E18. The multidose system of any one of embodiments E1-E17, wherein the ophthalmic composition has a pH of about 6.0 to about 8.5.
E19. The multidose system of any one of embodiments E1-E17, wherein the ophthalmic composition further comprises a buffering agent.
E20. The multidose system of embodiment E19, wherein the buffering agent is present at a concentration of about 0.001% w/v to about 2% w/v.
E21. An ophthalmic composition comprising:
   a therapeutic agent,
   a viscosifying agent at a concentration of about 0.01% w/v to about 5% w/v; and
   an alkyl aryl polyether alcohol surfactant at a concentration of about 0.005% w/v to about 7% w/v, wherein the composition lacks a preservative.
E22. The ophthalmic composition of embodiment E21, wherein the therapeutic agent is apraclonidine or olopatadine.
E23. The ophthalmic composition of embodiment E22, wherein the therapeutic agent is apraclonidine.
E24. The ophthalmic composition of embodiment E22, wherein the therapeutic agent is olopatadine.
E25. The ophthalmic composition of any one of embodiments E21-E24, wherein the therapeutic agent is present at a concentration of about 0.01% w/v to about 1% w/v.
E26. The ophthalmic composition of embodiment E25, wherein the therapeutic agent is present at a concentration of about 0.01% w/v to about 0.25% w/v.
E27. The ophthalmic composition of embodiment E26, wherein the therapeutic agent is present at a concentration of about 0.125% w/v.
E28. The ophthalmic composition of embodiment E25, wherein the therapeutic agent is present at a concentration of about 0.7% w/v.
E29. The ophthalmic composition of any one of embodiments E21-E28, wherein the viscosifying agent is present at a concentration of about 0.01% w/v to about 0.8% w/v.
E30. The ophthalmic composition of embodiment E29, wherein the viscosifying agent is present a concentration of about 0.3% w/v.
E31. The ophthalmic composition of embodiment E29, wherein the viscosifying agent is present at a concentration of about 0.4% w/v.
E32. The ophthalmic composition of embodiment E29, wherein the viscosifying agent is hydroxypropyl methylcellulose (HPMC).
E33. The ophthalmic composition of any one of embodiments E21-E32, wherein the surfactant is tyloxapol.
E34. The ophthalmic composition of any one of embodiments E21-E33, wherein the surfactant is present at a concentration of about 0.01% w/v to about 1% w/v.
E35. The ophthalmic composition of any one of embodiments E21-E34, wherein the ophthalmic composition has a pH of about 6.0 to about 8.5.
E36. The ophthalmic composition of any one of embodiments E21-E35, wherein the ophthalmic composition further comprises a buffering agent.
E37. The ophthalmic composition of embodiment E39, wherein the buffering agent is present at a concentration of about 0.001% w/v to about 2% w/v.
E38. A method of forming a multidose system comprising:
   receiving an ophthalmic composition comprising a therapeutic agent, a viscosifying agent at a concentration of about 0.01% w/v to about 0.5% w/v, and a surfactant at a concentration of about 0.01% w/v to about 7% w/v;
   dispensing a volume of the ophthalmic composition into a reservoir of an MDPF system;
   fastening an end piece onto the reservoir of the MDPF system; and
   sterilizing the MDPF system.
E39. The method of embodiment E38, wherein the volume is about 1 mL to about 15 mL.
E40. The method of embodiment E39, wherein the volume is about 5 mL.

Overall, the ophthalmic compositions and multidose systems of the present invention can provide maintained bioavailability benefits while concurrently reducing droplet spreading. A surfactant of the present disclosure causes the ophthalmic composition to be delivered at a consistent dosage/droplet size when using an MDPF dropper bottle or container, reducing the amount of droplet spreading. Additionally, due to the enhanced control of the dosing when using the surfactant, less overall ophthalmic composition can be used in order to provide sufficient dosing amounts of therapeutic agent, resulting in a lower cost to the consumer for the ophthalmic composition.

While the foregoing is directed to embodiments of the present disclosure, other and further embodiments of the disclosure may be devised without departing from the basic scope thereof, and the scope thereof is determined by the claims that follow.

What is claimed is:

1. A multidose system for topically administering a composition to the eye comprising:
a multidose preservative-free eye dropper bottle system; and
an ophthalmic composition comprising:
a therapeutic agent,
a viscosifying agent at a concentration of 0.01% w/v to 5% w/v; and
a surfactant at a concentration of 0.005% w/v to 7% w/,
wherein the composition lacks a preservative.

2. The multidose system of claim 1, wherein the therapeutic agent is apraclonidine or olopatadine.

3. The multidose system of claim 1, wherein the therapeutic agent is present at a concentration of 0.01% w/v to 1.0% w/v.

4. The multidose system of claim 1, wherein the viscosifying agent is present at a concentration of 0.01% w/v to 0.8% w/v.

5. The multidose system of claim 1, wherein the viscosifying agent is hydroxypropyl methylcellulose (HPMC).

6. The multidose system of claim 1, wherein the surfactant is selected from the group consisting of tyloxapol, polysorbate 80, and polyoxyl 40 stearate.

7. The multidose system of claim 1, wherein the surfactant is present at a concentration of 0.01% w/v to 1% w/v.

8. The multidose system of claim 1, wherein the ophthalmic composition has a pH of 6.0 to 8.5.

9. The multidose system of claim 1, wherein the ophthalmic composition further comprises a buffering agent.

10. The multidose system of claim 1, wherein the ophthalmic composition comprises an alkyl aryl polyether alcohol surfactant at a concentration of 0.01% w/v to 7% w/v.

11. The multidose system of claim 10, wherein the therapeutic agent is apraclonidine or olopatadine.

12. The multidose system of claim 10, wherein the therapeutic agent is present at a concentration of 0.01% w/v to 1% w/v.

13. The multidose system of claim 10, wherein the viscosifying agent is present at a concentration of 0.01% w/v to 0.8% w/v.

14. The multidose system of claim 10, wherein the viscosifying agent is hydroxypropyl methylcellulose (HPMC).

15. The multidose system of claim 10, wherein the alkyl aryl polyether alcohol surfactant is tyloxapol.

16. The multidose system of claim 10, wherein the alkyl aryl polyether alcohol surfactant is present a concentration of 0.01% w/v to 1% w/v.

17. The multidose system of claim 10, wherein the ophthalmic composition has a pH of 6.0 to 8.5.
